(19) United States
(12) Patent Application Publication
(10) Pub. No.: US 2026/0369374 A1
(43) Pub. Date: Nov. 19, 2026

(54) Method For Generating Medical Image Inspection Report

(71) Applicant: BEAMWORKS INC., Daegu (KR)

(72) Inventors: Dohwan KWAK, Gyeongsangbuk-do (KR); Won Hwa KIM, Daegu (KR); Jaeil KIM, Daegu (KR)

(73) Assignee: BEAMWORKS INC., Daegu (KR)

(21) Appl. No.: 19/476,104

(22) PCT Filed: Mar. 20, 2024
(86) PCT No.: PCT/KR2024/003469

§ 371 Date: Oct. 16, 2025

(30) Foreign Application Priority Data
Apr. 17, 2023 (KR) 10-2023-0049863

(51) Int. Cl. G16H 15/00; G06F 40/40; G06T 7/00; G06T 7/73; G06T 11/60

(52) U.S. Cl. CPC G16H 15/00; G06F 40/40; G06T 7/0012; G06T 7/73; G06T 11/60; G06T 2207/10132; G06T 2207/20084; G06T 2207/30096

(57) ABSTRACT

According to the present disclosure, a method for generating a medical imaging examination report, performed by a computing device may include: obtaining information on a lesion based on a medical image; generating a text report based on the information on the lesion; generating a visual report based on the information on the lesion; and automatically generating a corresponding modification in the visual report when the text report is modified, or automatically generating a corresponding modification in the text report when the visual report is modified.

### TECHNICAL FIELD

The present disclosure relates to a method for generating a medical imaging examination report, and more particularly, to a method for automatically generating an examination report on medical imaging such as ultrasound.

### BACKGROUND ART

After an ultrasound examination, a user (for example, a doctor) is essentially required to prepare a radiology report, which is a medical imaging examination report. In preparing the radiology report, the user must spend considerable time and personal effort, and the format and terminology used differ according to the user's preferences. In addition, before preparing the radiology report, the user must each time use a loading function in a picture archiving and communication system (PACS) to call up a preferred template form and then modify the entire contents to fit the corresponding exam, which is cumbersome.

Furthermore, a conventional radiology report is composed only of text and does not include visual information for identifying the location or characteristics of a lesion, thereby lacking visibility and communicability. A radiology report composed only of text has poor communicability when explaining to a patient without medical background knowledge, and there is a problem in that additional time and effort are required to find reference literature related to the lesion in the ultrasound image.

Korean Registered Patent No. 10-1014554 (Feb. 8, 2011) discloses a method for providing a preview page and an ultrasound system.

### DISCLOSURE

### Technical Problem

The present disclosure is directed to providing a technology for automatically generating a medical imaging examination report based on acquired medical imaging (for example, an ultrasound image). For example, the present disclosure is directed to providing a technology for automatically generating a text report and a visual report based on information on a lesion included in the medical imaging, and for automatically reflecting the same modification in the other report when a modification occurs in one report.

Meanwhile, a technical object to be achieved by the present disclosure is not limited to the above-mentioned technical object, and various technical objects can be included within the scope which is obvious to those skilled in the art from contents to be described below.

### Technical Solution

In order to achieve the above-described object, disclosed is a method for generating a medical imaging examination report, performed by a computing device. Here, the method may include: obtaining information on a lesion based on a medical image; generating a text report based on the information on the lesion; generating a visual report based on the information on the lesion; and automatically generating a corresponding modification in the visual report when the text report is modified, or automatically generating a corresponding modification in the text report when the visual report is modified.

In an embodiment, the medical image may comprise an ultrasound image, and the medical imaging examination report may comprise a radiology report.

In an embodiment, obtaining the information on the lesion based on the medical image may comprise: obtaining location information on the lesion based on the medical image; and obtaining characteristic information on the lesion.

In an embodiment, obtaining the location information on the lesion based on the medical image may comprise obtaining the location information on the lesion by inputting the medical image into an artificial intelligence model, and the location information on the lesion may comprise at least one of direction information, angle information, or distance information of the lesion.

In an embodiment, the lesion may be a lesion related to a breast, and obtaining the location information on the lesion by inputting the medical image into the artificial intelligence model may comprise at least one of: direction information of the breast where the lesion exists; angle information of the lesion with reference to a nipple; or distance information of the lesion with reference to the nipple.

In an embodiment, obtaining the characteristic information on the lesion may comprise obtaining the characteristic information on the lesion based on an output generated by artificial intelligence.

In an embodiment, the output generated by the artificial intelligence may be generated by an artificial intelligence model different from the artificial intelligence model utilized in obtaining the location information, and the output generated by the artificial intelligence may comprise at least one of: a file in which inference performed in advance is stored; or a real-time inference result generated by the different artificial intelligence model.

In an embodiment, the different artificial intelligence model may share at least a portion of hidden layers with the artificial intelligence model utilized in obtaining the location information.

In an embodiment, obtaining the characteristic information on the lesion may comprise at least one of: obtaining malignancy information of the lesion; obtaining direction information of the lesion; obtaining size information of the lesion; obtaining area information of the lesion; or obtaining shape information of the lesion.

In an embodiment, obtaining the malignancy information of the lesion may comprise obtaining Breast Imaging-Reporting and Data System (BI-RADS) category information.

In an embodiment, generating the text report may comprise generating the text report by inputting the location information on the lesion and the characteristic information on the lesion into a text report generation model, and the text report generation model may comprise a rule-based model or an artificial intelligence model.

In an embodiment, obtaining the information on the lesion based on the medical image may further comprise obtaining contour information on the lesion.

In an embodiment, generating the visual report may comprise generating the visual report by inputting the location information on the lesion, the characteristic information on the lesion, and the contour information on the lesion into a visual report generation model, and the visual report generation model may comprise a rule-based model or an artificial intelligence model.

In an embodiment, generating the visual report by inputting the location information on the lesion, the characteristic information on the lesion, and the contour information on the lesion into the visual report generation model may comprise: generating a lesion object based on the characteristic information on the lesion and the contour information on the lesion; and visually arranging the lesion object based on the location information on the lesion.

In an embodiment, generating the lesion object based on the characteristic information on the lesion and the contour information on the lesion may comprise at least one of: determining a visual shape of the lesion object based on at least one of direction information, size information, area information, or shape information of the lesion and the contour information on the lesion; or determining a color of the lesion object based on malignancy information of the lesion.

In an embodiment, automatically generating the corresponding modification in the visual report when the text report is modified may comprise: identifying modified information on the lesion based on modified text; and automatically generating a modified visual report by inputting the modified information on the lesion into a visual report generation model.

In an embodiment, identifying the modified information on the lesion based on the modified text may comprise identifying at least one of modified location information, modified direction information, modified size information, modified area information, modified shape information, or modified malignancy information on the lesion based on the modified text, and automatically generating the modified visual report by inputting the modified information on the lesion into the visual report generation model may comprise modifying at least one of a visual arrangement, a shape, or a color of a lesion object based on at least one of the modified location information, the modified direction information, the modified size information, the modified area information, the modified shape information, or the modified malignancy information on the lesion.

In an embodiment, automatically generating a corresponding modification in the text report when the visual report is modified may comprise: identifying modified information on the lesion based on modified visual information; and automatically generating a modified text report by inputting the modified information on the lesion into a text report generation model.

In an embodiment, identifying the modified information on the lesion based on the modified visual information may comprise identifying at least one of modified location information, modified direction information, modified size information, modified area information, modified shape information, or modified malignancy information on the lesion based on the modified visual information, and automatically generating the modified text report by inputting the modified information on the lesion into the text report generation model may comprise automatically modifying a text associated with at least one of the modified location information, the modified direction information, the modified size information, the modified area information, the modified shape information, or the modified malignancy information on the lesion.

According to one embodiment of the present disclosure, a computer program stored in a non-transitory computer-readable storage medium is disclosed. When the computer program is executed by one or more processors, the computer program causes the one or more processors to perform operations for generating a medical imaging examination report, the operations comprising: an operation of obtaining information on a lesion based on a medical image; an operation of generating a text report based on the information on the lesion; an operation of generating a visual report based on the information on the lesion; and an operation of automatically generating a corresponding modification in the visual report when the text report is modified, or automatically generating a corresponding modification in the text report when the visual report is modified.

In an embodiment, the operation of obtaining the information on the lesion based on the medical image may comprise: an operation of obtaining location information on the lesion based on the medical image; and an operation of obtaining characteristic information on the lesion.

In an embodiment, the operation of obtaining the location information on the lesion based on the medical image may comprise an operation of obtaining the location information on the lesion by inputting the medical image into an artificial intelligence model, and the location information on the lesion may comprise at least one of direction information, angle information, or distance information of the lesion.

In an embodiment, the operation of obtaining the characteristic information on the lesion may comprise an operation of obtaining the characteristic information on the lesion based on an output generated by artificial intelligence.

In an embodiment, the operation of generating the text report may comprise an operation of generating the text report by inputting the location information on the lesion and the characteristic information on the lesion into a text report generation model, and the text report generation model may comprise a rule-based model or an artificial intelligence model.

In an embodiment, the operation of obtaining the information on the lesion based on the medical image may further comprise an operation of obtaining contour information on the lesion.

In an embodiment, the operation of generating the visual report may comprise an operation of generating the visual report by inputting the location information on the lesion, the characteristic information on the lesion, and the contour information on the lesion into a visual report generation model, and the visual report generation model may comprise a rule-based model or an artificial intelligence model.

In an embodiment, the operation of automatically generating the corresponding modification in the visual report when the text report is modified may comprise: an operation of identifying modified information on the lesion based on modified text; and an operation of automatically generating a modified visual report by inputting the modified information on the lesion into a visual report generation model.

In an embodiment, the operation of automatically generating a corresponding modification in the text report when the visual report is modified may comprise: an operation of identifying modified information on the lesion based on modified visual information; and an operation of automatically generating a modified text report by inputting the modified information on the lesion into a text report generation model.

According to one embodiment of the present disclosure, a computing device is disclosed. The device may comprise at least one processor; and a memory, wherein the at least one processor may be configured to: obtain information on a lesion based on a medical image; generate a text report based on the information on the lesion; generate a visual report based on the information on the lesion; and automatically generate a corresponding modification in the visual report when the text report is modified, or automatically generate a corresponding modification in the text report when the visual report is modified.

In an embodiment, the at least one processor may be configured to identify modified information on the lesion based on modified text; and automatically generate a modified visual report by inputting the modified information on the lesion into a visual report generation model.

In an embodiment, the at least one processor may be configured to identify at least one of modified location information, modified direction information, modified size information, modified area information, modified shape information, or modified malignancy information on the lesion based on the modified text, and modify at least one of a visual arrangement, a shape, or a color of a lesion object based on at least one of the modified location information, the modified direction information, the modified size information, the modified area information, the modified shape information, or the modified malignancy information on the lesion.

In an embodiment, the at least one processor may be configured to identify modified information on the lesion based on modified visual information; and automatically generate a modified text report by inputting the modified information on the lesion into a text report generation model.

In an embodiment, the at least one processor may be configured to identify at least one of modified location information, modified direction information, modified size information, modified area information, modified shape information, or modified malignancy information on the lesion based on the modified visual information, and automatically modify a text associated with at least one of the modified location information, the modified direction information, the modified size information, the modified area information, the modified shape information, or the modified malignancy information on the lesion.

### Advantageous Effects

The present disclosure may provide a technology for automatically generating a medical imaging examination report based on acquired medical imaging (for example, ultrasound imaging). For example, the present disclosure may minimize a user's time and effort by automatically generating a text report and a visual report based on information on a lesion related to medical imaging.

In addition, the present disclosure may automatically generate a text report and a visual report related to medical imaging without prior input from a user.

Furthermore, the present disclosure may dynamically and automatically reflect the same modification in one report when a modification occurs in the other among the automatically generated text report and visual report.

Meanwhile, the effects of the present disclosure are not limited to the above-mentioned effects, and various effects can be included within the scope which is obvious to those skilled in the art from contents to be described below.

### DESCRIPTION OF DRAWINGS

FIG. 1 is a block diagram of a computing device for generating a medical imaging examination report according to an embodiment of the present disclosure.

FIG. 2 is a conceptual diagram illustrating a neural network according to an embodiment of the present disclosure.

FIG. 3 is a schematic flowchart of an operation of generating a medical imaging examination report in response to a modification according to an embodiment of the present disclosure.

FIG. 4 is a schematic diagram illustrating a main UI of a program for a medical imaging examination report according to an embodiment of the present disclosure.

FIG. 5 is a schematic diagram illustrating a program UI for a visual report according to an embodiment of the present disclosure.

FIG. 6 is a flowchart illustrating a method for generating a medical imaging examination report according to an embodiment of the present disclosure.

FIG. 7 is a simple and normal schematic diagram of an exemplary computing environment in which the embodiments of the present disclosure may be implemented.

### BEST MODE FOR INVENTION

Various exemplary embodiments will now be described with reference to drawings. In the present specification, various descriptions are presented to provide appreciation of the present disclosure. However, it is apparent that the exemplary embodiments can be executed without the specific description.

“Component”, “module”, “system”, and the like which are terms used in the specification refer to a computer-related entity, hardware, firmware, software, and a combination of the software and the hardware, or execution of the software. For example, the component may be a processing procedure executed on a processor, the processor, an object, an execution thread, a program, and/or a computer, but is not limited thereto. For example, both an application executed in a computing device and the computing device may be the components. One or more components may reside within the processor and/or a thread of execution. One component may be localized in one computer. One component may be distributed between two or more computers. Further, the components may be executed by various computer-readable media having various data structures, which are stored therein. The components may perform communication through local and/or remote processing according to a signal (for example, data transmitted from another system through a network such as the Internet through data and/or a signal from one component that interacts with other components in a local system and a distribution system) having one or more data packets, for example.

The term “or” is intended to mean not exclusive “or” but inclusive “or”. That is, when not separately specified or not clear in terms of a context, a sentence “X uses A or B” is intended to mean one of the natural inclusive substitutions. That is, the sentence “X uses A or B” may be applied to any of the case where X uses A, the case where X uses B, or the case where X uses both A and B. Further, it should be understood that the term “and/or” used in this specification designates and includes all available combinations of one or more items among enumerated related items.

It should be appreciated that the term “comprise” and/or “comprising” means presence of corresponding features and/or components. However, it should be appreciated that the term “comprises” and/or “comprising” means that presence or addition of one or more other features, components, and/or a group thereof is not excluded. Further, when not separately specified or it is not clear in terms of the context that a singular form is indicated, it should be construed that the singular form generally means “one or more” in this specification and the claims.

The term “at least one of A or B” should be interpreted to mean “a case including only A”, “a case including only B”, and “a case in which A and B are combined”.

Those skilled in the art need to recognize that various illustrative logical blocks, configurations, modules, circuits, means, logic, and algorithm steps described in connection with the exemplary embodiments disclosed herein may be additionally implemented as electronic hardware, computer software, or combinations of both sides. To clearly illustrate the interchangeability of hardware and software, various illustrative components, blocks, configurations, means, logic, modules, circuits, and steps have been described above generally in terms of their functionalities. Whether the functionalities are implemented as the hardware or software depends on a specific application and design restrictions given to an entire system. Skilled artisans may implement the described functionalities in various ways for each particular application. However, such implementation decisions should not be interpreted as causing a departure from the scope of the present disclosure.

The description of the presented exemplary embodiments is provided so that those skilled in the art of the present disclosure use or implement the present disclosure. Various modifications to the exemplary embodiments will be apparent to those skilled in the art. Generic principles defined herein may be applied to other embodiments without departing from the scope of the present disclosure. Therefore, the present disclosure is not limited to the exemplary embodiments presented herein. The present disclosure should be analyzed within the widest range which is coherent with the principles and new features presented herein.

In the present disclosure, a network function and an artificial neural network and a neural network may be interchangeably used.

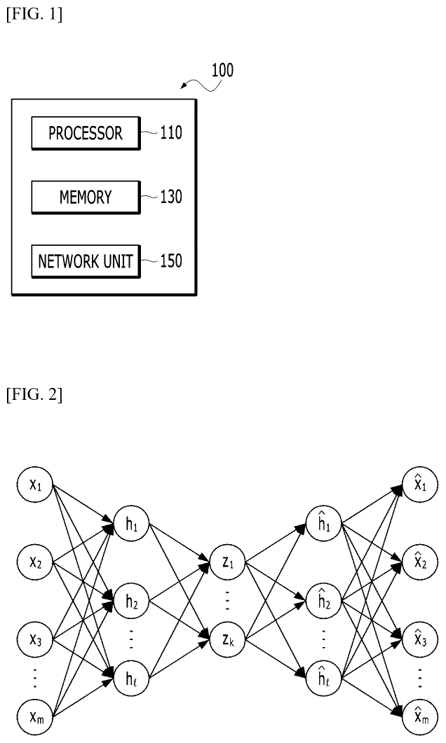

FIG. 1 is a block diagram of a computing device for generating a medical imaging examination report according to an embodiment of the present disclosure.

A configuration of the computing device 100 illustrated in FIG. 1 is only an example shown through simplification. In an exemplary embodiment of the present disclosure, the computing device 100 may include other components for performing a computing environment of the computing device 100 and only some of the disclosed components may constitute the computing device 100.

The computing device 100 may include a processor 110, a memory 130, and a network unit 150.

The processor 110 may be constituted by one or more cores and may include processors for data analysis and deep learning, which include a central processing unit (CPU), a general purpose graphics processing unit (GPGPU), a tensor processing unit (TPU), and the like of the computing device. The processor 110 may read a computer program stored in the memory 130 to perform data processing for machine learning according to an exemplary embodiment of the present disclosure. According to an exemplary embodiment of the present disclosure, the processor 110 may perform a calculation for learning the neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in an exemplary embodiment of the present disclosure, processors of a plurality of computing devices may be used together to process the learning of the network function and the data classification using the network function. Further, the computer program executed in the computing device according to an exemplary embodiment of the present disclosure may be a CPU, GPGPU, or TPU executable program.

According to an embodiment of the present disclosure, the memory (130) may store any type of information generated or determined by the processor (110), as well as any type of information received by the network unit (150).

According to an exemplary embodiment of the present disclosure, the memory 130 may include at least one type of storage medium of a flash memory type storage medium, a hard disk type storage medium, a multimedia card micro type storage medium, a card type memory (for example, an SD or XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The description of the memory is just an example and the present disclosure is not limited thereto.

The network unit (150) according to an embodiment of the present disclosure may employ various wired communication systems such as a Public Switched Telephone Network (PSTN), xDSL (x Digital Subscriber Line), RADSL (Rate Adaptive DSL), MDSL (Multi Rate DSL), VDSL (Very High Speed DSL), UADSL (Universal Asymmetric DSL), HDSL (High Bit Rate DSL), and a Local Area Network (LAN).

In addition, the network unit (150) described herein may employ various wireless communication systems such as CDMA (Code Division Multiple Access), TDMA (Time Division Multiple Access), FDMA (Frequency Division Multiple Access), OFDMA (Orthogonal Frequency Division Multiple Access), SC-FDMA (Single Carrier-FDMA), and other systems.

In the present disclosure, the network unit (150) may be configured in any communication mode, whether wired or wireless, and may include various communication networks such as a Local Area Network (LAN), a Personal Area Network (PAN), or a Wide Area Network (WAN). Moreover, the network may be a known World Wide Web (WWW) and may also use short-range wireless transmission technologies such as Infrared Data Association (IrDA) or Bluetooth.

The technologies described in the present specification can be applied not only to the networks mentioned above but also to other types of networks.

FIG. 2 is a conceptual diagram illustrating a neural network according to an embodiment of the present disclosure.

Throughout the present specification, a computation model, the neural network, a network function, and the neural network may be used as the same meaning. The neural network may be generally constituted by an aggregate of calculation units which are mutually connected to each other, which may be called nodes. The nodes may also be called neurons. The neural network is configured to include one or more nodes. The nodes (alternatively, neurons) constituting the neural networks may be connected to each other by one or more links.

In the neural network, one or more nodes connected through the link may relatively form the relationship between an input node and an output node. Concepts of the input node and the output node are relative and a predetermined node which has the output node relationship with respect to one node may have the input node relationship in the relationship with another node and vice versa. As described above, the relationship of the input node to the output node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable and the weight is variable by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form a relationship of the input node and output node in the neural network. A characteristic of the neural network may be determined according to the number of nodes, the number of links, correlations between the nodes and the links, and values of the weights granted to the respective links in the neural network. For example, when the same number of nodes and links exist and there are two neural networks in which the weight values of the links are different from each other, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed through for reaching the corresponding node from the initial input node. However, a definition of the layer is predetermined for description and the order of the layer in the neural network may be defined by a method different from the aforementioned method. For example, the layers of the nodes may be defined by the distance from a final output node.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean nodes constituting the neural network other than the initial input node and the final output node.

In the neural network according to an exemplary embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another exemplary embodiment of the present disclosure, the number of nodes of the input layer may be smaller than the number of nodes of the output layer, and the neural network may be a neural network of a type in which the number of nodes decreases from the input layer to the hidden layer. Further, in the neural network according to yet another exemplary embodiment of the present disclosure, the number of nodes of the input layer may be larger than the number of nodes of the output layer, and the neural network may be a neural network of a type in which the number of nodes increases from the input layer to the hidden layer. The neural network according to still yet another exemplary embodiment of the present disclosure may be a neural network of a type in which the neural networks are combined.

A deep neural network (DNN) may refer to a neural network that includes a plurality of hidden layers in addition to the input and output layers. When the deep neural network is used, the latent structures of data may be determined. That is, latent structures of photos, text, video, voice, and music (e.g., what objects are in the photo, what the content and feelings of the text are, what the content and feelings of the voice are) may be determined. The deep neural network may include a convolutional neural network (CNN), a recurrent neural network (RNN), an auto encoder, generative adversarial networks (GAN), a restricted Boltzmann machine (RBM), a deep belief network (DBN), a Q network, a U network, a Siam network, a Generative Adversarial Network (GAN), and the like. The description of the deep neural network described above is just an example and the present disclosure is not limited thereto.

In an exemplary embodiment of the present disclosure, the network function may include the auto encoder. The auto encoder may be a kind of artificial neural network for outputting output data similar to input data. The auto encoder may include at least one hidden layer and odd hidden layers may be disposed between the input and output layers. The number of nodes in each layer may be reduced from the number of nodes in the input layer to an intermediate layer called a bottleneck layer (encoding), and then expanded symmetrical to reduction to the output layer (symmetrical to the input layer) in the bottleneck layer. The auto encoder may perform non-linear dimensional reduction. The number of input and output layers may correspond to a dimension after preprocessing the input data. The auto encoder structure may have a structure in which the number of nodes in the hidden layer included in the encoder decreases as a distance from the input layer increases. When the number of nodes in the bottleneck layer (a layer having a smallest number of nodes positioned between an encoder and a decoder) is too small, a sufficient amount of information may not be delivered, and as a result, the number of nodes in the bottleneck layer may be maintained to be a specific number or more (e.g., half of the input layers or more).

The neural network may be learned in at least one scheme of supervised learning, unsupervised learning, semi supervised learning, or reinforcement learning. The learning of the neural network may be a process in which the neural network applies knowledge for performing a specific operation to the neural network.

The neural network may be learned in a direction to minimize errors of an output. The learning of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weight of each node of the neural network. In the case of the supervised learning, the learning data labeled with a correct answer is used for each learning data (i.e., the labeled learning data) and in the case of the unsupervised learning, the correct answer may not be labeled in each learning data. That is, for example, the learning data in the case of the supervised learning related to the data classification may be data in which category is labeled in each learning data. The labeled learning data is input to the neural network, and the error may be calculated by comparing the output (category) of the neural network with the label of the learning data. As another example, in the case of the unsupervised learning related to the data classification, the learning data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the learning data may be generally a subset of actual data (i.e., data to be processed using the learned neural network), and as a result, there may be a learning cycle in which errors for the learning data decrease, but the errors for the actual data increase. Overfitting is a phenomenon in which the errors for the actual data increase due to excessive learning of the learning data. For example, a phenomenon in which the neural network that learns a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the learning data, regularization, dropout of omitting a part of the node of the network in the process of learning, utilization of a batch normalization layer, etc., may be applied.

The present disclosure relates to a technology in which, when a specific examination case of a patient is selected, the acquired medical image is analyzed and a medical imaging examination report (for example, a text report and a visual report) is automatically generated. A conventional method of preparing a medical imaging examination report (for example, a radiology report) has entirely relied on the effort and time investment of a user (for example, a doctor), and the format and terminology used have also depended on the preference and discretion of the user. In contrast, the present disclosure, unlike the conventional method of preparing a medical imaging examination report, does not require prior input from the user, allows the generated text report or visual report to be modified when necessary, and enables modifications in one of the text report or the visual report to be automatically reflected in the other.

The present disclosure can minimize modification and effort of the user by automatically generating and providing the medical imaging examination report in a standardized format. In addition, although understanding a medical imaging examination report composed only of text requires medical background knowledge, time, and effort, the present disclosure provides the effect of enabling intuitive understanding of the location, size, and malignancy of a lesion even with insufficient medical background knowledge, by expressing the text report as a visual report and providing them together. Accordingly, the present disclosure enables the user (for example, a doctor) to more clearly convey information on a lesion to a patient even when explaining the lesion by utilizing the medical imaging examination report generated based on the foregoing description.

The medical image according to an embodiment of the present disclosure may include various types of medical imaging such as an ultrasound image, a CT image, an X-ray image, an MRI image, or a nuclear medicine image. In addition, the medical imaging examination report may include various types of reports prepared based on images acquired by medical imaging equipment such as X-ray, CT, MRI, ultrasound, or nuclear medicine examination. Furthermore, the lesion may include various types of lesions related to a part of a patient's body (for example, brain, breast, organs, etc.).

Hereinafter, for convenience of explanation, an embodiment will be described in which the medical image relates to an ultrasound image, the medical imaging examination report includes a radiology report, and the lesion is a lesion related to a breast. In other words, the following embodiment describes a method of automatically generating a radiology report, which is a medical imaging examination report, based on an ultrasound image acquired by imaging a breast using an ultrasound device. However, the present disclosure is not limited thereto, and various embodiments may be included within the scope of the present disclosure.

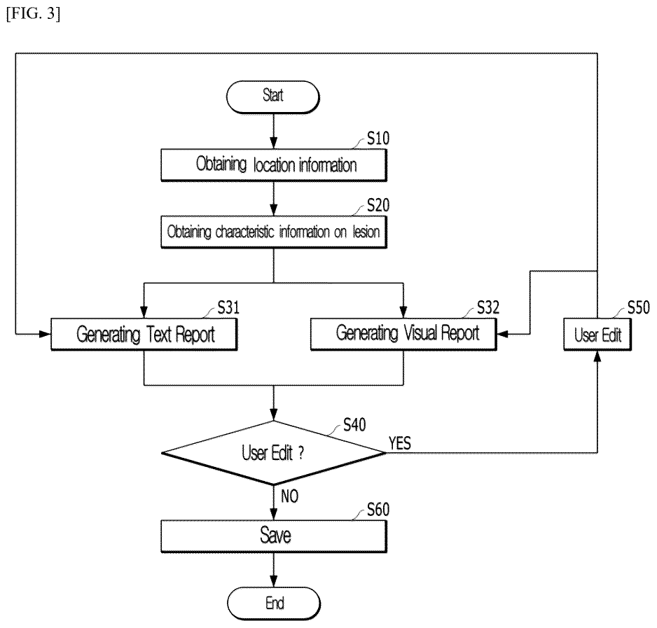

FIG. 3 is a schematic flowchart of an operation of generating a medical imaging examination report in response to a modification according to an embodiment of the present disclosure.

The operation of generating a medical imaging examination report according to an embodiment of the present disclosure may include a step S10 of obtaining location information, a step S20 of obtaining characteristic information on a lesion, a step S31 of generating a text report, a step S32 of generating a visual report, a step S40 of determining whether a user modification is made, a step S50 of receiving input information related to a user change, and a step S60 of storing. Steps S10 to S60 may, according to implementations of the present application, be further divided into additional steps or combined into fewer steps. In addition, some steps may be omitted as necessary, and the order among the steps may be changed.

According to an embodiment of the present disclosure, a computing device 100 may obtain information on a lesion based on a medical image. For example, the computing device 100 may obtain information on a lesion related to a breast based on an ultrasound image. In one example, the information on the lesion may include location information on the lesion, characteristic information on the lesion, and contour information on the lesion.

First, the computing device 100 may obtain location information on the lesion based on the medical image S10. For example, the computing device 100 may obtain the location information on the lesion by inputting the medical image into an artificial intelligence model. Here, the location information on the lesion may include at least one of direction information on the lesion (for example, direction information of a body part where the lesion is located), angle information, or distance information. In one example, when the lesion is a lesion related to a breast, the computing device 100 may obtain location information on the lesion including at least one of direction information of the breast where the lesion exists, angle information of the lesion with reference to a nipple, or distance information of the lesion with reference to the nipple. According to an embodiment, the computing device 100 may obtain direction information on whether the lesion exists in a left breast or a right breast. In addition, the computing device 100 may obtain angle information on the lesion with reference to a nipple in each of the left or right breasts, for example, which direction among a direction from 0 o'clock to 12 o'clock the lesion is located. In addition, the computing device 100 may obtain distance information considering a degree of separation (for example, cm) with reference to the nipple.

Next, the computing device 100 can obtain characteristic information on the lesion S20. For example, the computing device 100 may obtain the characteristic information on the lesion based on an output generated by artificial intelligence.

Here, the output generated by the artificial intelligence may be generated by a different artificial intelligence model than the artificial intelligence model utilized in obtaining the location information.

For reference, the output generated by the artificial intelligence may include at least one of a file in which inference performed in advance is stored or a real-time inference result generated by the different artificial intelligence model. For example, the computing device 100 may read a file in which inference results performed in advance by the different artificial intelligence model (e.g., an artificial intelligence model trained to predict characteristic information on a plurality of lesions) are stored to obtain characteristic information on the lesion.

In one embodiment, the file may be a JSON file in which corresponding patient information and characteristic information on the lesion are stored. In addition, the computing device 100 may directly input the obtained medical image into the different artificial intelligence model to obtain characteristic information on the lesion as a real-time inference result.

Meanwhile, the different artificial intelligence model may share at least a portion of hidden layers with the artificial intelligence model utilized in obtaining the location information.

In other words, from the viewpoint of “predicting information on the lesion,” since the task (Task) of the different artificial intelligence model may be similar to that of the artificial intelligence model utilized in obtaining the location information, the different artificial intelligence model may share at least a portion of hidden layers with the artificial intelligence model utilized in obtaining the location information.

Therefore, through such multitask learning (Multitask learning), the performance of predictions of the artificial intelligence model utilized in obtaining the location information and of the different artificial intelligence model can be improved.

In addition, the characteristic information may include at least one of malignancy information of the lesion, direction information of the lesion (e.g., arrangement direction information of the lesion itself), size information of the lesion, area information of the lesion, or shape information of the lesion, and may include various types of information in addition to such information.

In one embodiment, with respect to the malignancy information of the lesion, the computing device 100 may obtain the malignancy information of the lesion based on Breast Imaging-Reporting and Data System (BI-RADS) category information.

The Breast Imaging-Reporting and Data System (BI-RADS) category information is an index developed to standardize evaluation of lesions for breast cancer.

The computing device 100 may determine the malignancy information of the lesion based on the Breast Imaging-Reporting and Data System (BI-RADS) category information as at least one of Category 1 (negative), Category 2 (benign finding), Category 3 (probably benign), Category 4 (suspicious abnormality), Category 5 (highly suggestive of malignancy), or Category 6 (known biopsy-proven malignancy). In addition, the direction information of the lesion included in the characteristic information may include arrangement direction information of the lesion itself.

For example, the direction information of the lesion may include direction information indicating a direction toward which a specific portion included in the lesion is oriented and direction information indicating a direction toward which a central axis of the lesion is oriented. In addition, the size or area information of the lesion may include size or area information expressed at various scales. In addition, the shape information of the lesion may include various shape information including external appearance information of the lesion and shape information of a specific portion of the lesion.

Next, the computing device 100 can generate a text report based on the information on the lesion S31.

In one embodiment, the computing device 100 may generate the text report by inputting the location information on the lesion and the characteristic information on the lesion into a text report generation model. Here, the text report generation model may include a rule-based model or an artificial intelligence model.

For example, the computing device 100 may provide in text, based on the location information on the lesion, direction information of a breast where the lesion exists, angle information of the lesion with reference to a nipple (e.g., positioned at a direction of 11 o'clock to 12 o'clock with reference to the right nipple), or distance information of the lesion with reference to the nipple (e.g., positioned in a region spaced 3 cm from the nipple).

In addition, the computing device 100 may provide in text malignancy information of the lesion (e.g., information on a category classified in Breast Imaging-Reporting and Data System category information), direction information of the lesion, size information of the lesion, area information of the lesion, and shape information of the lesion.

Meanwhile, the computing device 100 can automatically generate a structured text report without prior input of a user. In addition, the text report generated by the text report generation model may minimize deviation in terms and templates of the text report depending on the user by additionally using structured text (e.g., medical information for reference on the lesion).

Next, the computing device 100 can generate a visual report based on the information on the lesion S32.

For example, the computing device 100 may generate the visual report by inputting the location information on the lesion, the characteristic information on the lesion, and the contour information on the lesion into a visual report generation model. Here, the visual report generation model may include a rule-based model or an artificial intelligence model.

More specifically, the computing device 100 may generate a lesion object based on the characteristic information on the lesion and the contour information on the lesion to generate the visual report. Here, the lesion object may be an image object generated based on the characteristic information on the lesion and the contour information on the lesion. In addition, the computing device 100 may determine a visual shape of the lesion object based on at least one of the direction information, size information, area information, or shape information of the lesion and the contour information on the lesion. In addition, the computing device 100 may generate lesion objects having different sizes, areas, and shapes corresponding to respective lesions based on at least one of the direction information, size information, area information, or shape information of the lesion and the contour information on the lesion.

For reference, the contour information on the lesion is information that is additionally utilized in the process of generating the visual report (and is not utilized in the process of generating the text report). By additionally utilizing such contour information on the lesion, external appearance information or boundary information of the lesion (which is difficult to express in the text report) can be generated in a clearer form and can be included in the visual report in a more clearly visualized state. Meanwhile, the contour information on the lesion may be generated together by a model for generating the characteristic information on the lesion, or may be generated by a model different from the model for generating the characteristic information on the lesion.

In addition, the computing device 100 may determine a color of the lesion object based on malignancy information of the lesion. For example, the computing device 100 may determine the color of the lesion object corresponding to Breast Imaging-Reporting and Data System (BI-RADS) category information classified into Category 1 (negative), Category 2 (benign finding), Category 3 (probably benign), Category 4 (suspicious abnormality), Category 5 (highly suggestive of malignancy), and Category 6 (known biopsy-proven malignancy). For example, the computing device 100 may determine a first color corresponding to Category 1, determine a second color corresponding to Category 2, determine a third color corresponding to Category 3, determine a fourth color corresponding to Category 4, determine a fifth color corresponding to Category 5, and determine a sixth color corresponding to Category 6.

In addition, the computing device 100 may visually arrange the lesion object based on the location information on the lesion. The computing device 100 may visually arrange the lesion object in consideration of the direction information, angle information, and distance information of the lesion, which are the location information on the lesion.

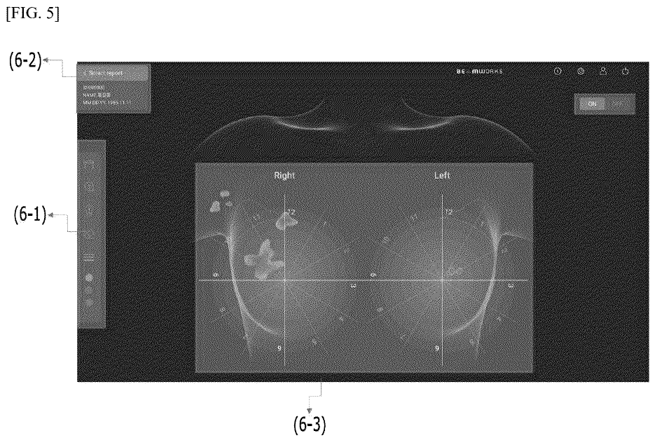

For example, referring to FIG. 5, a virtual region may be a shaped region of a graph formed in the shape of a breast in consideration of the direction information, angle information, and distance information of the lesion, which are the location information, in order to provide more accurate information. As an example, referring to FIG. 5, the computing device 100 may generate the visual report by overlaying the lesion object, whose visual shape has been determined, on the virtual region associated with at least a portion of the body.

According to one embodiment of the present disclosure, the computing device 100 may provide the generated text report and visual report through a user interface. A user may check the text report and the visual report output through the user interface and may perform changes (e.g., addition, modification, deletion, etc.) with respect to items requiring modification in the text report or the visual report. First, the computing device 100 may provide the generated text report and the visual report to the user interface. In addition, the computing device 100 may determine whether at least one of the generated text report and the visual report is modified by a user S40. At this time, if modification input information is not received, the computing device 100 may store the corresponding text report and visual report in association with patient information S60.

Meanwhile, the computing device 100 may receive at least one of modification input information for the text report or modification input information for the visual report from the user interface S50. If the modification input information received from the user interface is a modification of the text report, the computing device 100 may provide the modification input information to the text report generation step S31. In addition, if the modification input information received from the user interface is a modification of the visual report, the computing device 100 may provide the user modification input information to the visual report generation step S32.

According to one embodiment of the present disclosure, if the text report is modified, the computing device 100 may automatically generate a corresponding modification in the visual report. In addition, if the visual report is modified, the computing device 100 may automatically generate a corresponding modification in the text report.

In one embodiment, if the text report is modified, the computing device 100 may automatically generate a corresponding modification in the visual report based on “modified text.” More specifically, the computing device 100 may identify modified information on the lesion based on the “modified text.” For example, the computing device 100 may identify at least one of modified location information, modified direction information, modified size information, modified area information, modified shape information, or modified malignancy information on the lesion based on the “modified text.” For example, if the modified text information is “3 cm from the nipple→changed to 2.5 cm from the nipple,” the computing device 100 may identify modified information on the lesion as modified location information on the lesion based on the “modified text.”

Thereafter, the computing device 100 may input the modified information on the lesion (modified text) into a visual report generation model to automatically generate a modified visual report. For example, the computing device 100 may modify at least one of a visual arrangement, a shape, or a color of a lesion object based on at least one of the modified location information, the modified direction information, the modified size information, the modified area information, the modified shape information, or the modified malignancy information on the lesion. For example, if the modified text information is “modified location information on the lesion (e.g., 3 cm from the nipple→changed to 2.5 cm from the nipple),” the computing device 100 may input the modified location information on the lesion into the visual report generation model to automatically generate a modified visual report. In other words, the computing device 100 may automatically generate a modified visual report in which the location of the lesion object is arranged at 2.5 cm from the nipple based on the modified text information.

In one embodiment, if the visual report is modified, the computing device 100 may automatically generate a corresponding modification in the text report. More specifically, the computing device 100 may identify modified information on the lesion based on modified visual information. For example, the computing device 100 may identify at least one of modified location information, modified direction information, modified size information, modified area information, modified shape information, or modified malignancy information on the lesion based on the “modified visual information.” For example, if the modified visual information is “color of the lesion object changed from a third color→to a fourth color,” the computing device 100 may identify modified information on the lesion as malignancy information of the lesion based on the “modified visual information.”

In addition, the computing device 100 may input the modified information on the lesion (modified visual information) into a text report generation model to automatically generate a modified text report. For example, the computing device 100 may automatically modify a text associated with at least one of the modified location information, the modified direction information, the modified size information, the modified area information, the modified shape information, or the modified malignancy information on the lesion. For example, if the modified visual information is “malignancy information on the lesion (e.g., color of the lesion object changed from a third color→to a fourth color),” the computing device 100 may input the modified malignancy information on the lesion into the text report generation model to automatically generate a modified text report. In other words, the computing device 100 may automatically generate a modified text report in which “malignancy information on the lesion: Category 3” is changed to “malignancy information on the lesion: Category 4” in the text report based on the “modified visual information.”

According to one embodiment of the present disclosure, the computing device 100 may update the automatically generated modified text report or modified visual report based on the text report or the visual report and may provide the updated report through a user interface. A user may check the modified (updated) text report or visual report, and when a change occurs, may provide modification input information to the computing device 100 through the user interface. On the other hand, if no additional change occurs, the computing device 100 may store the modified text report or the modified visual report in association with patient information S60.

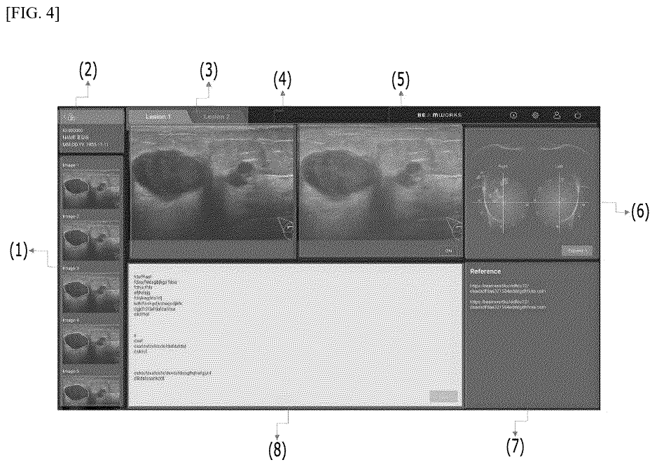

FIG. 4 is a schematic diagram illustrating a main UI of a program for a medical imaging examination report according to an embodiment of the present disclosure.

Referring to FIG. 4, a program main UI for a medical imaging examination report may include a plurality of obtained medical images 1, patient information 2, lesion information 3, a B-mode image 4, artificial intelligence model analysis results 5, a visual report 6, a text report 7, and a reference 8 on the corresponding lesion.

For example, the plurality of obtained medical images 1 may be images obtained when a freeze mode is activated in an ultrasound probe. Here, the freeze mode may be a mode that stops and stores a screen when a still screen is needed for storage or description of an ultrasound image for a lesion site. In addition, the plurality of medical images 1 may be images obtained from the ultrasound probe at predetermined time intervals (e.g., one-second intervals). However, the present disclosure is not limited thereto, and the plurality of medical images 1 may include images obtained using various medical devices. In addition, the patient information 2 may include a patient's unique identification ID, name, date of birth, age, and information on the date on which the image is obtained. In addition, the lesion information 3 may include information on the lesion obtained by artificial intelligence. In addition, the B-mode image 4, also referred to as B (brightness) mode, may be an image in which a cross-section of an organ is displayed on a screen in real time in black-and-white shading. In addition, the artificial intelligence model analysis results 5 may include results generated by inputting location information on the lesion, characteristic information on the lesion, and contour information on the lesion into a visual report generation model. For example, the artificial intelligence model analysis results 5 may be a B-mode image overlaid with a heatmap generated by inputting the information into the visual report generation model. In addition, the visual report 6 may include results in which a lesion object generated based on the characteristic information on the lesion and the contour information on the lesion is visually arranged based on the location information on the lesion, considering results generated by inputting the location information on the lesion, the characteristic information on the lesion, and the contour information on the lesion into the visual report generation model. In addition, the text report 7 may include results generated by inputting the location information on the lesion and the characteristic information on the lesion into a text report generation model. In addition, the reference 8 on the corresponding lesion may include reference literature related to information on the lesion based on the generated text report or visual report. The text report and visual report shown in FIG. 4 may be generated from the same operation as the text report and visual report described earlier with reference to FIG. 3. Even though the contents are omitted hereinafter, the descriptions of the text report and the visual report are equally applicable to the text report and the visual report illustrated in FIG. 4.

FIG. 5 is a schematic diagram illustrating a program UI for a visual report according to an embodiment of the present disclosure. For reference, FIG. 5 may be a result of enlarging a visual report to a full screen by using an extension function in a program main UI for a medical imaging examination report.

For example, referring to FIG. 5, a program UI for the visual report may include a toolbar 6-1, patient information 6-2, and lesion object visualization information 6-3. For example, the toolbar 6-1 may provide a pen drawing or erasing function that allows color selection and line type selection, and may provide a distance measuring tool. At this time, a shape generated by using the toolbar 6-1 (for example, drawing with a pen, etc.) is not related to modification of the generated visual report, and may simply be used for description and annotation purposes. In addition, the patient information 2 may include a patient's unique identification ID, name, date of birth, age, and information on the date on which the image is obtained. In addition, the lesion information 3 may include information on the lesion obtained by artificial intelligence.

The lesion object visualization information 6-3 may be a result of visually arranging a lesion object generated based on characteristic information on the lesion and contour information on the lesion based on location information on the lesion. The visual report illustrated in FIG. 5 may be generated from the same operation as the visual report described earlier with reference to FIG. 3. Even though the contents are omitted hereinafter, the descriptions of the visual report are equally applicable to the visual report illustrated in FIG. 5.

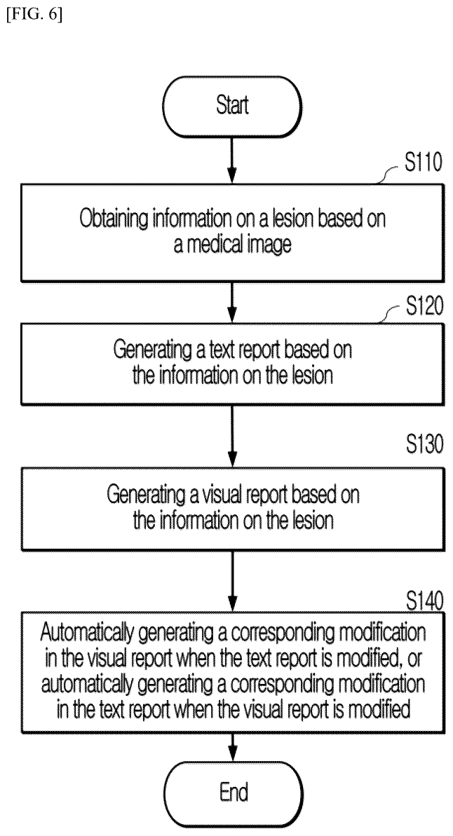

FIG. 6 is a flowchart illustrating a method for generating a medical imaging examination report according to an embodiment of the present disclosure.

The method for generating a medical imaging examination report illustrated in FIG. 6 may be performed by the computing device 100. Hereinafter, even though not specifically mentioned, the above descriptions regarding the computing device 100 are equally applicable to the description of the method for generating a medical imaging examination report.

Referring to FIG. 6, the method for generating a medical imaging examination report according to one embodiment of the present disclosure may include: a step S110 of obtaining information on a lesion based on a medical image; a step S120 of generating a text report based on the information on the lesion; a step S130 of generating a visual report based on the information on the lesion; and a step S140 of automatically generating a corresponding modification in the visual report when the text report is modified, or automatically generating a corresponding modification in the text report when the visual report is modified. In addition, the method for generating a medical imaging examination report according to one embodiment of the present disclosure may be performed by the computing device 100.

The step S110 is a step of obtaining information on a lesion based on a medical image. For example, the medical image may include an ultrasound image, and the medical imaging examination report may include a radiology report. Such step S110 may include a step of obtaining location information on the lesion based on the medical image and a step of obtaining characteristic information on the lesion.

The step S120 is a step of generating a text report based on the information on the lesion. Such step S120 may include a step of generating the text report by inputting the location information on the lesion and the characteristic information on the lesion into a text report generation model, and the text report generation model may include a rule-based model or an artificial intelligence model.

The step S130 is a step of generating a visual report based on the information on the lesion. Such step S130 may include a step of generating the visual report by inputting the location information on the lesion, the characteristic information on the lesion, and the contour information on the lesion into a visual report generation model, and the visual report generation model may include a rule-based model or an artificial intelligence model.

The step S140 is a step of automatically generating a corresponding modification in the visual report when the text report is modified, or automatically generating a corresponding modification in the text report when the visual report is modified. Such step S140 may include an operation of identifying modified information on the lesion based on modified text and automatically generating a modified visual report by inputting the modified information on the lesion into a visual report generation model. In addition, the step S140 may include an operation of identifying modified information on the lesion based on modified visual information and automatically generating a modified text report by inputting the modified information on the lesion into a text report generation model.

The steps mentioned in the above description may, according to implementations of the present disclosure, be further divided into additional steps or combined into fewer steps. In addition, some steps may be omitted as necessary, and the order of the steps may also be changed.

Disclosed is a computer readable medium storing the data structure according to an exemplary embodiment of the present disclosure. The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection relationship between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device can perform operations while using the resources of the computing device to a minimum. Specifically, the computing device can increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO—Last in First Out) in which the data is input last and output first. The queue is a data listing structure that may access data limitedly and unlike a stack, the queue may be a data structure (FIFO—First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

Throughout the present specification, a computation model, the neural network, a network function, and the neural network may be used as the same meaning. Hereinafter, the computation model, the neural network, the network function, and the neural network will be integrated and described as the neural network. The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an active function associated with each node or layer of the neural network, and a loss function for learning the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an active function associated with each node or layer of the neural network, and a loss function for learning the neural network or a combination thereof. In addition to the above-described configurations, the data structure including the neural network may include predetermined other information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called nodes. The nodes may also be called neurons. The neural network is configured to include one or more nodes.

The data structure may include data input into the neural network. The data structure including the data input into the neural network may be stored in the computer readable medium. The data input to the neural network may include learning data input in a neural network learning process and/or input data input to a neural network in which learning is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example and the present disclosure is not limited thereto.

The data structure may include the weight of the neural network (in the present disclosure, the weight and the parameter may be used as the same meaning). In addition, the data structures, including the weight of the neural network, may be stored in the computer readable medium. The neural network may include a plurality of weights. The weight may be variable and the weight is variable by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example and the present disclosure is not limited thereto.

As a non-limiting example, the weight may include a weight which varies in the neural network learning process and/or a weight in which neural network learning is completed. The weight which varies in the neural network learning process may include a weight at a time when a learning cycle starts and/or a weight that varies during the learning cycle. The weight in which the neural network learning is completed may include a weight in which the learning cycle is completed. Accordingly, the data structure including the weight of the neural network may include a data structure including the weight which varies in the neural network learning process and/or the weight in which neural network learning is completed. Accordingly, the above-described weight and/or a combination of each weight are included in a data structure including a weight of a neural network. The data structure is just an example and the present disclosure is not limited thereto.

The data structure including the weight of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weight of the serialized neural network may be reconfigured in the same computing device or another computing device through deserialization. The data structure including the weight of the neural network is not limited to the serialization. Furthermore, the data structure including the weight of the neural network may include a data structure (for example, B-Tree, Trie, m-way search tree, AVL tree, and Red-Black Tree in a nonlinear data structure) to increase the efficiency of operation while using resources of the computing device to a minimum. The above-described matter is just an example and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of learning cycle iterations, weight initialization (for example, setting a range of weight values to be subjected to weight initialization), and Hidden Unit number (e.g., the number of hidden layers and the number of nodes in the hidden layer). The data structure is just an example and the present disclosure is not limited thereto.

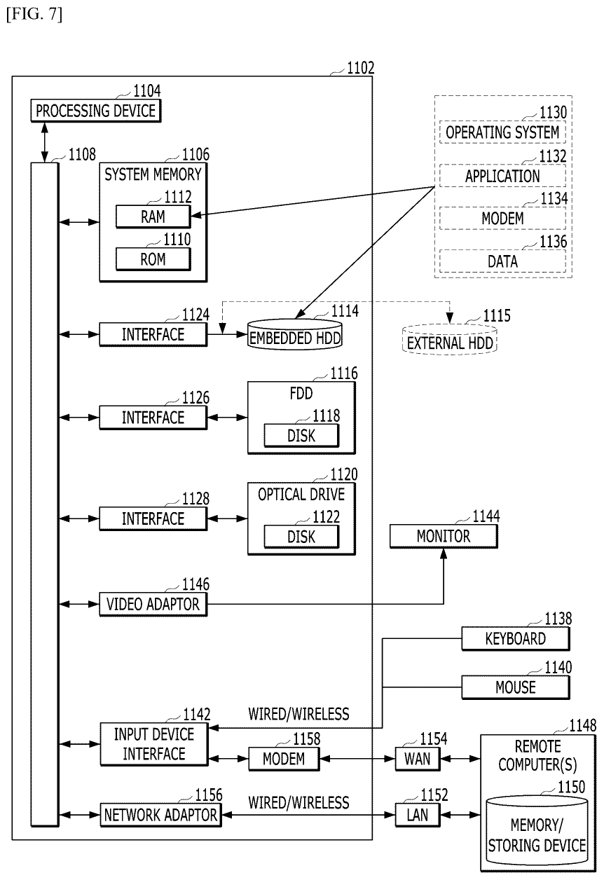

FIG. 7 is a normal and schematic view of an exemplary computing environment in which the exemplary embodiments of the present disclosure may be implemented. It is described above that the present disclosure may be generally implemented by the computing device, but those skilled in the art will well know that the present disclosure may be implemented in association with a computer executable command which may be executed on one or more computers and/or in combination with other program modules and/or a combination of hardware and software.

In general, the program module includes a routine, a program, a component, a data structure, and the like that execute a specific task or implement a specific abstract data type. Further, it will be well appreciated by those skilled in the art that the method of the present disclosure can be implemented by other computer system configurations including a personal computer, a handheld computing device, microprocessor-based or programmable home appliances, and others (the respective devices may operate in connection with one or more associated devices as well as a single-processor or multi-processor computer system, a mini computer, and a main frame computer.

The exemplary embodiments described in the present disclosure may also be implemented in a distributed computing environment in which predetermined tasks are performed by remote processing devices connected through a communication network. In the distributed computing environment, the program module may be positioned in both local and remote memory storage devices.

The computer generally includes various computer readable media. Media accessible by the computer may be computer readable media regardless of types thereof and the computer readable media include volatile and non-volatile media, transitory and non-transitory media, and mobile and non-mobile media. As a non-limiting example, the computer readable media may include both computer readable storage media and computer readable transmission media. The computer readable storage media include volatile and non-volatile media, transitory and non-transitory media, and mobile and non-mobile media implemented by a predetermined method or technology for storing information such as a computer readable instruction, a data structure, a program module, or other data. The computer readable storage media include a RAM, a ROM, an EEPROM, a flash memory or other memory technologies, a CD-ROM, a digital video disk (DVD) or other optical disk storage devices, a magnetic cassette, a magnetic tape, a magnetic disk storage device or other magnetic storage devices or predetermined other media which may be accessed by the computer or may be used to store desired information, but are not limited thereto.

The computer readable transmission media generally implement the computer readable command, the data structure, the program module, or other data in a carrier wave or a modulated data signal such as other transport mechanism and include all information transfer media. The term “modulated data signal” means a signal acquired by setting or changing at least one of characteristics of the signal so as to encode information in the signal. As a non-limiting example, the computer readable transmission media include wired media such as a wired network or a direct-wired connection and wireless media such as acoustic, RF, infrared and other wireless media. A combination of any media among the aforementioned media is also included in a range of the computer readable transmission media.

An exemplary environment 1100 that implements various aspects of the present disclosure including a computer 1102 is shown and the computer 1102 includes a processing device 1104, a system memory 1106, and a system bus 1108. The system bus 1108 connects system components including the system memory 1106 (not limited thereto) to the processing device 1104. The processing device 1104 may be a predetermined processor among various commercial processors. A dual processor and other multi-processor architectures may also be used as the processing device 1104.

The system bus 1108 may be any one of several types of bus structures which may be additionally interconnected to a local bus using any one of a memory bus, a peripheral device bus, and various commercial bus architectures. The system memory 1106 includes a read only memory (ROM) 1110 and a random access memory (RAM) 1112. A basic input/output system (BIOS) is stored in the non-volatile memories 1110 including the ROM, the EPROM, the EEPROM, and the like and the BIOS includes a basic routine that assists in transmitting information among components in the computer 1102 at a time such as in-starting. The RAM 1112 may also include a high-speed RAM including a static RAM for caching data, and the like.

The computer 1102 also includes an interior hard disk drive (HDD) 1114 (for example, EIDE and SATA), in which the interior hard disk drive 1114 may also be configured for an exterior purpose in an appropriate chassis (not illustrated), a magnetic floppy disk drive (FDD) 1116 (for example, for reading from or writing in a mobile diskette 1118), and an optical disk drive 1120 (for example, for reading a CD-ROM disk 1122 or reading from or writing in other high-capacity optical media such as the DVD, and the like). The hard disk drive 1114, the magnetic disk drive 1116, and the optical disk drive 1120 may be connected to the system bus 1108 by a hard disk drive interface 1124, a magnetic disk drive interface 1126, and an optical drive interface 1128, respectively. An interface 1124 for implementing an exterior drive includes at least one of a universal serial bus (USB) and an IEEE 1394 interface technology or both of them.

The drives and the computer readable media associated therewith provide non-volatile storage of the data, the data structure, the computer executable instruction, and others. In the case of the computer 1102, the drives and the media correspond to storing of predetermined data in an appropriate digital format. In the description of the computer readable media, the mobile optical media such as the HDD, the mobile magnetic disk, and the CD or the DVD are mentioned, but it will be well appreciated by those skilled in the art that other types of media readable by the computer such as a zip drive, a magnetic cassette, a flash memory card, a cartridge, and others may also be used in an exemplary operating environment and further, the predetermined media may include computer executable commands for executing the methods of the present disclosure.

Multiple program modules including an operating system 1130, one or more application programs 1132, other program module 1134, and program data 1136 may be stored in the drive and the RAM 1112. All or some of the operating system, the application, the module, and/or the data may also be cached in the RAM 1112. It will be well appreciated that the present disclosure may be implemented in operating systems which are commercially usable or a combination of the operating systems.

A user may input instructions and information in the computer 1102 through one or more wired/wireless input devices, for example, pointing devices such as a keyboard 1138 and a mouse 1140. Other input devices (not illustrated) may include a microphone, an IR remote controller, a joystick, a game pad, a stylus pen, a touch screen, and others. These and other input devices are often connected to the processing device 1104 through an input device interface 1142 connected to the system bus 1108, but may be connected by other interfaces including a parallel port, an IEEE 1394 serial port, a game port, a USB port, an IR interface, and others.

A monitor 1144 or other types of display devices are also connected to the system bus 1108 through interfaces such as a video adapter 1146, and the like. In addition to the monitor 1144, the computer generally includes other peripheral output devices (not illustrated) such as a speaker, a printer, others.

The computer 1102 may operate in a networked environment by using a logical connection to one or more remote computers including remote computer(s) 1148 through wired and/or wireless communication. The remote computer(s) 1148 may be a workstation, a computing device computer, a router, a personal computer, a portable computer, a micro-processor based entertainment apparatus, a peer device, or other general network nodes and generally includes multiple components or all of the components described with respect to the computer 1102, but only a memory storage device 1150 is illustrated for brief description. The illustrated logical connection includes a wired/wireless connection to a local area network (LAN) 1152 and/or a larger network, for example, a wide area network (WAN) 1154. The LAN and WAN networking environments are general environments in offices and companies and facilitate an enterprise-wide computer network such as Intranet, and all of them may be connected to a worldwide computer network, for example, the Internet.

When the computer 1102 is used in the LAN networking environment, the computer 1102 is connected to a local network 1152 through a wired and/or wireless communication network interface or an adapter 1156. The adapter 1156 may facilitate the wired or wireless communication to the LAN 1152 and the LAN 1152 also includes a wireless access point installed therein in order to communicate with the wireless adapter 1156. When the computer 1102 is used in the WAN networking environment, the computer 1102 may include a modem 1158 or has other means that configure communication through the WAN 1154 such as connection to a communication computing device on the WAN 1154 or connection through the Internet. The modem 1158 which may be an internal or external and wired or wireless device is connected to the system bus 1108 through the serial port interface 1142. In the networked environment, the program modules described with respect to the computer 1102 or some thereof may be stored in the remote memory/storage device 1150. It will be well known that an illustrated network connection is exemplary and other means configuring a communication link among computers may be used.

The computer 1102 performs an operation of communicating with predetermined wireless devices or entities which are disposed and operated by the wireless communication, for example, the printer, a scanner, a desktop and/or a portable computer, a portable data assistant (PDA), a communication satellite, predetermined equipment or place associated with a wireless detectable tag, and a telephone. This at least includes wireless fidelity (Wi-Fi) and Bluetooth wireless technology. Accordingly, communication may be a predefined structure like the network in the related art or just ad hoc communication between at least two devices.

The wireless fidelity (Wi-Fi) enables connection to the Internet, and the like without a wired cable. The Wi-Fi is a wireless technology such as the device, for example, a cellular phone which enables the computer to transmit and receive data indoors or outdoors, that is, anywhere in a communication range of a base station. The Wi-Fi network uses a wireless technology called IEEE 802.11 (a, b, g, and others) in order to provide safe, reliable, and high-speed wireless connection. The Wi-Fi may be used to connect the computers to each other or the Internet and the wired network (using IEEE 802.3 or Ethernet). The Wi-Fi network may operate, for example, at a data rate of 11 Mbps (802.11a) or 54 Mbps (802.11b) in unlicensed 2.4 and 5 GHz wireless bands or operate in a product including both bands (dual bands).

It will be appreciated by those skilled in the art that information and signals may be expressed by using various different predetermined technologies and techniques. For example, data, instructions, commands, information, signals, bits, symbols, and chips which may be referred in the above description may be expressed by voltages, currents, electromagnetic waves, magnetic fields or particles, optical fields or particles, or predetermined combinations thereof.

It may be appreciated by those skilled in the art that various exemplary logical blocks, modules, processors, means, circuits, and algorithm steps described in association with the exemplary embodiments disclosed herein may be implemented by electronic hardware, various types of programs or design codes (for easy description, herein, designated as software), or a combination of all of them. In order to clearly describe the intercompatibility of the hardware and the software, various exemplary components, blocks, modules, circuits, and steps have been generally described above in association with functions thereof. Whether the functions are implemented as the hardware or software depends on design restrictions given to a specific application and an entire system. Those skilled in the art of the present disclosure may implement functions described by various methods with respect to each specific application, but it should not be interpreted that the implementation determination departs from the scope of the present disclosure.

Various exemplary embodiments presented herein may be implemented as manufactured articles using a method, a device, or a standard programming and/or engineering technique. The term manufactured article includes a computer program, a carrier, or a medium which is accessible by a predetermined computer-readable storage device. For example, a computer-readable storage medium includes a magnetic storage device (for example, a hard disk, a floppy disk, a magnetic strip, or the like), an optical disk (for example, a CD, a DVD, or the like), a smart card, and a flash memory device (for example, an EEPROM, a card, a stick, a key drive, or the like), but is not limited thereto. Further, various storage media presented herein include one or more devices and/or other machine-readable media for storing information.

It will be appreciated that a specific order or a hierarchical structure of steps in the presented processes is one example of exemplary accesses. It will be appreciated that the specific order or the hierarchical structure of the steps in the processes within the scope of the present disclosure may be rearranged based on design priorities. Appended method claims provide elements of various steps in a sample order, but the method claims are not limited to the presented specific order or hierarchical structure.

The description of the presented exemplary embodiments is provided so that those skilled in the art of the present disclosure use or implement the present disclosure. Various modifications of the exemplary embodiments will be apparent to those skilled in the art and general principles defined herein can be applied to other exemplary embodiments without departing from the scope of the present disclosure. Therefore, the present disclosure is not limited to the exemplary embodiments presented herein, but should be interpreted within the widest range which is coherent with the principles and new features presented herein.

### Mode for Invention

Related contents in the best mode for carrying out the present disclosure are described as above.

### Industrial Applicability

The present disclosure may be used in a computing device, a system, etc., for predicting a structure of a protein by using a twist-based structure update.

1. A method for generating a medical imaging examination report, performed by a computing device, the method comprising:
obtaining information on a lesion based on a medical image;
generating a text report based on the information on the lesion;
generating a visual report based on the information on the lesion; and
automatically generating a corresponding modification in the visual report when the text report is modified, or automatically generating a corresponding modification in the text report when the visual report is modified.

2. The method of claim 1, wherein the medical image comprises an ultrasound image, and the medical imaging examination report comprises a radiology report.

3. The method of claim 1, wherein obtaining information on the lesion based on the medical image comprises:
obtaining location information on the lesion based on the medical image; and
obtaining characteristic information on the lesion.

4. The method of claim 3, wherein obtaining the location information on the lesion based on the medical image comprises:
obtaining the location information on the lesion by inputting the medical image into an artificial intelligence model, and
wherein the location information on the lesion comprises at least one of direction information, angle information, or distance information of the lesion.

5. The method of claim 4, wherein the lesion is a lesion related to a breast, and
wherein obtaining the location information on the lesion by inputting the medical image into the artificial intelligence model comprises at least one of:
direction information of the breast where the lesion exists;
angle information of the lesion with reference to a nipple; or
distance information of the lesion with reference to the nipple.

6. The method of claim 3, wherein obtaining the characteristic information on the lesion comprises:
obtaining the characteristic information on the lesion based on an output generated by artificial intelligence.

7. The method of claim 6, wherein the output generated by the artificial intelligence is generated by an artificial intelligence model different from the artificial intelligence model utilized in obtaining the location information, and wherein the output generated by the artificial intelligence comprises at least one of:
a file in which inference performed in advance is stored; or
a real-time inference result generated by the different artificial intelligence model.

8. The method of claim 7, wherein the different artificial intelligence model shares at least a portion of hidden layers with the artificial intelligence model utilized in obtaining the location information.

9. The method of claim 3, wherein obtaining the characteristic information on the lesion comprises at least one of:
obtaining malignancy information of the lesion;
obtaining direction information of the lesion;
obtaining size information of the lesion;
obtaining area information of the lesion; or
obtaining shape information of the lesion.

10. The method of claim 9, wherein obtaining the malignancy information of the lesion comprises:
obtaining Breast Imaging-Reporting and Data System (BI-RADS) category information.

11. The method of claim 3, wherein generating the text report comprises:
generating the text report by inputting the location information on the lesion and the characteristic information on the lesion into a text report generation model, and
wherein the text report generation model comprises a rule-based model or an artificial intelligence model.

12. The method of claim 3, wherein obtaining the information on the lesion based on the medical image further comprises:
obtaining contour information on the lesion.

13. The method of claim 12, wherein generating the visual report comprises:
generating the visual report by inputting the location information on the lesion, the characteristic information on the lesion, and the contour information on the lesion into a visual report generation model, and
wherein the visual report generation model comprises a rule-based model or an artificial intelligence model.

14. The method of claim 13, wherein generating the visual report by inputting the location information on the lesion, the characteristic information on the lesion, and the contour information on the lesion into the visual report generation model comprises:
generating a lesion object based on the characteristic information on the lesion and the contour information on the lesion; and
visually arranging the lesion object based on the location information on the lesion.

15. The method of claim 13, wherein generating the lesion object based on the characteristic information on the lesion and the contour information on the lesion comprises at least one of:
determining a visual shape of the lesion object based on at least one of direction information, size information, area information, or shape information of the lesion and the contour information on the lesion; or
determining a color of the lesion object based on malignancy information of the lesion.

16. The method of claim 1, wherein automatically generating the corresponding modification in the visual report when the text report is modified comprises:
identifying modified information on the lesion based on a modified text; and
automatically generating a modified visual report by inputting the modified information on the lesion into a visual report generation model.

17. The method of claim 16, wherein identifying the modified information on the lesion based on the modified text comprises:
identifying at least one of modified location information, modified direction information, modified size information, modified area information, modified shape information, or modified malignancy information on the lesion based on the modified text, and
wherein automatically generating the modified visual report by inputting the modified information on the lesion into the visual report generation model comprises:
modifying at least one of a visual arrangement, a shape, or a color of a lesion object based on at least one of the modified location information, the modified direction information, the modified size information, the modified area information, the modified shape information, or the modified malignancy information on the lesion.

18. The method of claim 1, wherein automatically generating a corresponding modification in the text report when the visual report is modified comprises:
identifying modified information on the lesion based on modified visual information; and
automatically generating a modified text report by inputting the modified information on the lesion into a text report generation model.

19. The method of claim 18, wherein identifying the modified information on the lesion based on the modified visual information comprises:
identifying at least one of modified location information, modified direction information, modified size information, modified area information, modified shape information, or modified malignancy information on the lesion based on the modified visual information, and
wherein automatically generating the modified text report by inputting the modified information on the lesion into the text report generation model comprises:
automatically modifying a text associated with at least one of the modified location information, the modified direction information, the modified size information, the modified area information, the modified shape information, or the modified malignancy information on the lesion.

20. A computer program stored in a non-transitory computer-readable storage medium, wherein when the computer program is executed by one or more processors, the computer program causes the one or more processors to perform operations for generating a medical imaging examination report, the operations comprising:
an operation of obtaining information on a lesion based on a medical image;
an operation of generating a text report based on the information on the lesion;
an operation of generating a visual report based on the information on the lesion; and
an operation of automatically generating a corresponding modification in the visual report when the text report is modified, or automatically generating a corresponding modification in the text report when the visual report is modified.

21. (canceled)

22. (canceled)

23. (canceled)

24. (canceled)

25. (canceled)

26. (canceled)

27. (canceled)

28. (canceled)

29. A computing device comprising:
at least one processor; and
a memory,
wherein the at least one processor is configured to:
obtain information on a lesion based on a medical image;
generate a text report based on the information on the lesion;
generate a visual report based on the information on the lesion; and
automatically generate a corresponding modification in the visual report when the text report is modified, or automatically generate a corresponding modification in the text report when the visual report is modified.

30. (canceled)

31. (canceled)

32. (canceled)

33. (canceled)
